SEQUENCE LISTING (2 sequences)

SEQ ID NO: 1 - protein, 370 aa, Artificial Sequence
  NKG2D-CD8ahinge-CD8TM-4-1BB-CD3??
DYKDDDDKGGGGSFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - DNA, 1113 bp, Artificial Sequence
  NKG2D-CD8ahinge-CD8TM-4-1BB-CD3??
gattacaaggatgacgacgataagggtggtggtggttctttcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

(19) United States
(12) Patent Application Publication
(10) Pub. No.: US 2020/0360437 A1
(43) Pub. Date: Nov. 19, 2020

(54) CHIMERIC ANTIGEN RECEPTOR, NKG2D CAR-NK CELLS EXPRESSING THE CHIMERIC ANTIGEN RECEPTOR, AND PREPARATION AND APPLICATION THEREOF

(71) Applicant: Asclepius (Suzhou) Technology Company Group Co., Ltd., Suzhou (CN)

(72) Inventors: Kunkun HAN, Suzhou (CN); Jianmin FU, Suzhou (CN); Huashun LI, Suzhou (CN)

(21) Appl. No.: 16/986,749

(22) Filed: Aug. 6, 2020

Related U.S. Application Data
(63) Continuation of application No. PCT/CN2019/074683, filed on Feb. 3, 2019.

(30) Foreign Application Priority Data
Feb. 7, 2018 (CN) 201810123851.3
Jan. 31, 2019 (CN) 201910100288.2

(51) Int. Cl. A61K 35/17; C07K 16/30; C07K 14/705; C07K 14/725; A61P 35/00

(52) U.S. Cl. CPC A61K 35/17; C07K 16/3023; C07K 16/3015; C07K 16/3046; A61K 9/0019; C07K 14/7051; C07K 14/70578; A61P 35/00; C07K 14/70517

(57) ABSTRACT

The present invention provides a chimeric antigen receptor, a NKG2D CAR-NK cell expressing the chimeric antigen receptor, and preparation and application thereof. The chimeric antigen receptor comprising an antigen binding domain, a transmembrane domain and a costimulatory signal transduction region, and the antigen binding domain can specifically bind to tumor specific antigen of NKG2D ligands and can activate NK cells through the transmembrane domain and the costimulatory signal transduction region. The NKG2D CAR-NK cell provided by the present invention use NKG2D ligands as target antigens, and can specifically kill tumor cells. It can be used as a therapeutic drug for tumor diseases, for the treatment of tumor with highly expressing of ligands of NKG2D molecule, thus providing new treatments for the tumors.

### CROSS-REFERENCE TO RELATED APPLICATION

This application is a continuation application of International patent application No. PCT/CN2019/074683, filed on Feb. 3, 2019 which claims priority to Chinese patent application No. CN201810123851.3, filed on Feb. 7, 2018, and claims priority to Chinese patent application No. CN201910100288.2, filed on Jan. 31, 2019. All of the aforementioned patent applications are hereby incorporated by reference in their entireties.

### TECHNICAL FIELD

The present invention relates to the field of biological medicine, in particular to a chimeric antigen receptor, a NKG2D CAR-NK cell expressing the chimeric antigen receptor, and preparation and application thereof.

### BACKGROUND

Chimeric antigen receptor (abbreviated CAR) modified immune cells is to use genetic engineering methods to modify immune cells, which makes the immune cells express exogenous anti-tumor gene. The CAR gene mainly includes an extracellular recognition domain and an intracellular signal transduction domain: wherein the former is used to recognize tumor surface specific molecules, and the latter is used to initiate immune cell responses after the recognize of the tumor surface molecules and exert cytotoxicity. The CAR mainly uses T-cells as carriers. However, in treatment of tumors with CAR-T cells, the levels of IL-6 and other cytokines will increase dramatically, resulting in cytokine release syndrome phenomenon and problems such as on-target/off-target toxicity and neurotoxicity, which will endanger the lives of patients in severe cases. Moreover, T cells must be isolated out of the body (this process is time-consuming and expensive). Furthermore, considering the T cells are modified for specific patients, however some patients may not be competent for collection of T cells or do not have enough time to wait for the preparation process of T cells. Although nowadays the CAR-T is developing towards a universal CAR-T, which also increases the clinical risk and the difficulty of operation. In addition, to face the high cost of CAR-T, these limitations may lead to that some patients who are expected to benefit from CAR-T immunotherapy cannot be administered.

Natural killer (abbreviated NK) cells are an important part of the non-specific immune system, and the key mediator cells of the innate immune system. NK cells are a kind of broad spectrum immune cells, which has the specific function of rapidly discovering and destroying abnormal cells (such as cancer cells or virus infected cells), and can demonstrate strong ability of dissolving abnormal cells without requiring being sensitized in advance or HLA matching. Using immune cells (including NK cells) to treat cancer is a new trend in recent years. This new therapy is expected to be promising for the treatment of tumors that are refractory to traditional surgery, chemotherapy and radiotherapy.

NKG2D is an activating receptor on NK cells, which can recognize MHC-I molecules, and play an important role in innate immunity. NKG2D is involved in the recognition of virus-infected cells and the killing of tumor cells on NK. NKG2D belongs to the family of C-type lectin-like receptors, and the receptor encoded by this gene is present in the natural killer group2 (NKG2) complex. The NKG2 gene complex is located on human chromosome 12. NKG2D is a type II transmembrane protein. NKG2D needs to complete signal transduction through the charged residues of the transmembrane region binding to some adaptor proteins. Human NKG2D can associate with an activating protein (DAP10) of 10 kDa DNAX. DAP10 has a YXXM motif (Tyr-X-X-Meth) within the cytoplasm, which can recruit phosphatidylinositol trihydroxy kinase (PI3K) and growth factor receptor-bound protein-2. NKG2D is expressed by all NK cells, most NKT cells, and macrophages. In addition, NKG2D is also present on the surface of CD8+ T cells. Under certain circumstances, NKG2D is not expressed by human and mouse CD4+ T cells. However, in patient's body, NKG2D-expressing CD4+ T cells mainly accumulate in tumor tissues. NKG2D can bind to many different ligands that belong to major histocompatibility complex class I (MHC-I)-related proteins. MIC-A and MIC-B are another family of human NKG2D ligands. Both MIC-A and MIC-B are polymorphic. Currently, MIC-A has 61 alleles and MIC-B has 30 alleles. The ligands of NKG2D molecules are not expressed or expressed at low levels in normal cells, but when the cells are infected or cancerous, the expression levels of these ligands will increase significantly.

At present, there aren't CAR-NK cells for the treatment of tumor with expressing of NKG2D and related diseases, but there are only CAR-T cells for the treatment of tumor with expressing of NKG2D and related diseases, however, they have high toxic side effects and high cost. If we can develop and study a kind of NKG2D CAR-NK cell, which will inevitably promote the progress in the field of tumor therapy.

### SUMMARY

In view of this, the present invention provides a chimeric antigen receptor, a NKG2D CAR-NK cell expressing the chimeric antigen receptor, and preparation and application thereof. The cell can specifically recognize and kill tumor, and has more effective tumor killing activity.

The present invention provides a chimeric antigen receptor comprising an antigen binding domain, a transmembrane domain and a costimulatory signal transduction region, wherein the antigen binding domain can specifically bind to tumor specific antigen NKG2D ligands, and activate NK cells through the transmembrane domain and the costimulatory signal transduction region.

Illustratively, the antigen binding domain contains NKG2D, and the NKG2D can specifically bind to NKG2D ligands.

Illustratively, the NKG2D ligands are selected from one or more of major histocompatibility complex class I (MHC-I), MIC-A and MIC-B.

Illustratively, the transmembrane domain is selected from one or more of CD28, CD3ε, CD45, CD4, CD5, CD8, CD9, CD16, CD22, CD33, CD37, CD134, CD137, ICOS and CD154, preferably, the transmembrane domain is CD8 transmembrane domain, and/or,

the costimulatory signal transduction region contains the intracellular domain of costimulatory molecule, which is selected from one or more of CD3ζ, CD3γ, CD3δ, CD3ε, CD5, CD22, CD79a, CD79b, CD66d, CD2, CD4, CD5, CD28, CD134, CD137, ICOS, CD154, 4-1BB and OX40.

Preferably, the costimulatory signal transduction region contains the intracellular domains of 4-1BB and CD3ζ.

Illustratively, the chimeric antigen receptor is a fusion protein with a structure of NKG2D-CD8αhinge-CD8™-4-1BB-CD3ζ.

Illustratively, the amino acid sequence of the fusion protein NKG2D-CD8αhinge-CD8™-4-1BB-CD3ζ is shown in SEQ ID NO: 1 or homologous sequence thereof and so on.

Illustratively, the homologous sequence has the identity of about 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, 99.9% or more to the original sequence.

Another aspect of the present invention provides a nucleotide sequence which encodes the above-mentioned chimeric antigen receptor.

Illustratively, the nucleotide sequence is shown in SEQ ID NO: 2 or degenerate sequence thereof and so on.

Illustratively, the degenerate sequence has the identity of about 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, 99.9% or more to the original sequence.

The present invention also provides a NKG2D CAR-NK cell, and the NKG2D CAR-NK cell can express a chimeric antigen receptor, wherein the chimeric antigen receptor comprises an antigen binding domain, a transmembrane domain and a costimulatory signal transduction region, and the antigen binding domain can specifically bind to tumor specific antigen NKG2D ligands and can activate NK cells through the transmembrane domain and the costimulatory signal transduction region.

Illustratively, the antigen binding domain contains NKG2D, and the NKG2D can specifically bind NKG2D ligands.

Illustratively, the NKG2D ligands are selected from one or more of major histocompatibility complex class I (MHC-I), MIC-A and MIC-B.

Illustratively, the transmembrane domain is selected from one or more of CD28, CD3ε, CD45, CD4, CDS, CD8, CD9, CD16, CD22, CD33, CD37, CD134, CD137, ICOS and CD154; preferably, the transmembrane domain is CD8 transmembrane domain; and/ or, the costimulatory signal transduction region contains the intracellular domain of costimulatory molecule, which is selected from one or more of CD3ζ, CD3γ, CD3δ, CD3ε, CD5, CD22, CD79a, CD79b, CD66d, CD2, CD4, CDS, CD28, CD134, CD137, ICOS, CD154, 4-1BB and OX40; preferably, the costimulatory signal transduction region contains the intracellular domains of 4-1BB and CD3ζ.

Illustratively, the chimeric antigen receptor is a fusion protein with a structure of NKG2D-CD8αhinge-CD8™-4-1BB-CD3ζ.

Illustratively, the amino acid sequence of the fusion protein NKG2D-CD8αhinge-CD8™-4-1BB-CD3ζ is shown in SEQ ID NO: 1 or homologous sequence thereof and so on.

Illustratively, the homologous sequence has the identity of about 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, 99.9% or more to the original sequence.

In a specific embodiment of the present invention, the encoding nucleotide of the fusion protein NKG2D-CD8αhinge-CD8™-4-1BB-CD3ζ is shown in SEQ ID NO: 2 or homologous sequences thereof.

Illustratively, the homologous sequence has the identity of about 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, 99.9% or more to the original sequence.

In a specific embodiment of the invention, the NKG2D CAR-NK cell can effectively destroy and/or kill lung cancer cell, colorectal cancer cell, or breast cancer cell.

Another aspect of the present invention provides a preparation method of the above-mentioned NKG2D CAR-NK cells, wherein it includes the following steps:

(1) synthesizing and amplifying the above-mentioned nucleotide sequence, and cloning the nucleotide sequence into a lentiviral expression vector;

(2) infecting 293T cells with a lentiviral packaging plasmid and the lentiviral expression vector plasmid prepared by step (1), packaging and preparing a lentivirus;

(3) infecting NK-92 cells with the lentivirus prepared in step (2) to obtain NKG2D CAR-NK cells.

Illustratively, in the step (1), synthesizing and amplifying the encoding nucleotide sequence of fusion protein NKG2D-CD8αhinge-CD8™-4-1BB-CD3ζ;

The present invention also provides a use of the above-mentioned NKG2D CAR-NK cells in the preparation of medicaments for treating and/or preventing cancer.

Illustratively, the cancer is a tumor with highly expressing of NKG2D ligands and related diseases.

The present invention also provides a pharmaceutical composition comprising the above-mentioned NKG2D CAR-NK cells, and optional pharmaceutically acceptable adjuvants.

Illustratively, the effector-to-target ratio of NKG2D CAR-NK cells to tumor cells in the pharmaceutical composition is 0.5:1 to 1:1.

Illustratively, the dosage form of the pharmaceutical composition is aqua.

The present invention also provides a method for treating and/or preventing cancer by using the above-mentioned NKG2D CAR-NK cells, including administering an effective amount of drug containing NKG2D CAR-NK cells to patients needing treatment.

Illustratively, the cancer is a tumor with highly expressing of NKG2D ligands and related diseases.

Illustratively, the cancer is lung cancer, breast cancer, or colorectal cancer.

Illustratively, the dosage of NKG2D CAR-NK cells is 1.0×10⁶ cells/time to 10×10⁶ cells/time, preferably, the dosage is 2.5×10⁶ cells/time to 5.0×10⁶ cells/time.

Illustratively, the administration mode of NKG2D CAR-NK cells is intratumoral injection, intravenous injection, intrathoracic injection or local intervention.

Illustratively, the administration mode of the NKG2D CAR-NK cells is intravenous injection.

The chimeric antigen receptor NKG2D CAR-NK cells provided by the present invention can specifically bind to NKG2D ligands of tumor specific antigen, and activate NK cells through the transmembrane domain and the costimulatory signal transduction region. The NKG2D CAR-NK cells was constructed through using NKG2D (NKG2D receptor) for CAR-NK cells. The chimeric antigen receptor and NKG2D CAR-NK cells with expressing of the chimeric antigen receptor that use NKG2D ligands as target antigen, which can be used as a therapeutic agent for tumor diseases, for the treatment of tumor with highly expressing of NKG2D ligands, thus providing a new method for the prevention and treatment of tumor; it can improve its targeting ability by transforming NK92 cells with CAR, moreover, greatly improve its anti-tumor ability while increasing anti-tumor targeting sites NKG2D; in addition, by transforming NK92 cells with CAR, the NKG2D CAR-NK cells constructed by the present invention can not only have significantly improved targeting ability, but also have increased its anti-tumor activities; that is to say, the NKG2D CAR-NK cells were firstly constructed in the present invention, by transforming NK92 cells with CAR, they not only increase its targeting ability, but also get higher safety, lower toxicity and side effects and lower cost by transforming NK cells; and NKG2D CAR-NK cells have significantly improved targeting ability through the CAR cooperating with NKG2D, moreover, the NKG2D CAR-NK cells can be more effective in killing tumors compared with NK92 cells, and further expand the broad spectrum of using CAR-NK cells to treat and prevent tumors.

### BRIEF DESCRIPTION OF DRAWINGS

FIG. 1 shows the schematic construction map of the lentiviral vector PRRLSIN-NKG2D provided in Example.

FIGS. 2a to 2b show the results of positive rate of CAR cells of NKG2D CAR-NK detected by flow cytometry provided in Example, wherein FIG. 2a is the control group, and FIG. 2b is the experimental group.

FIGS. 3a to 3e show the results of the expression of NKG2D ligand MIC-A and MIC-B molecules in different tumor cells detected by conventional flow cytometry provided in Example, wherein FIG. 3a is an experimental result of lung cancer cell A549, FIG. 3b is an experimental result of lung cancer cell H1299, FIG. 3c is an experimental result of breast cancer cell MCF-7, FIG. 3d is an experimental result of breast cancer cell MDMB-231, FIG. 3e is an experimental result of colorectal cancer cell SW480.

FIGS. 4a to 4e show the experimental results of the specific lysis activity of NKG2D CAR -NK cells on the different tumor cells provided in Example, wherein FIG. 4a is an experimental result of lung cancer cell A549, FIG. 4b is an experimental result of lung cancer cell H1299, FIG. 4c is an experimental result of breast cancer cell MCF-7, FIG. 4d is an experimental result of breast cancer cell MDMB-231, FIG. 4e is an experimental result of colorectal cancer cell SW480.

FIGS. 5a to 5b show the tumor growth curves of the NPG mice treated with NKG2D CAR-NK cells provided in Example, wherein FIG. 5a is a tumor growth curve of the mice with a dosage of 2.5×10⁶ cells/mouse, FIG. 5b is a tumor growth curve of the mice with a dosage of 5×10⁶ cells/mouse.

FIG. 6 shows a result of the tumor inhibition rate of the NPG mice treated with NKG2D CAR-NK cells provided in Example.

FIG. 7 shows a result of the tumor weight of the NKP mice treated with NKG2D-CAR NK92 cells provided in Example.

FIG. 8 shows a result of the tumor weight percentage of the NKP mice treated with NKG2D-CAR NK92 cells provided in Example.

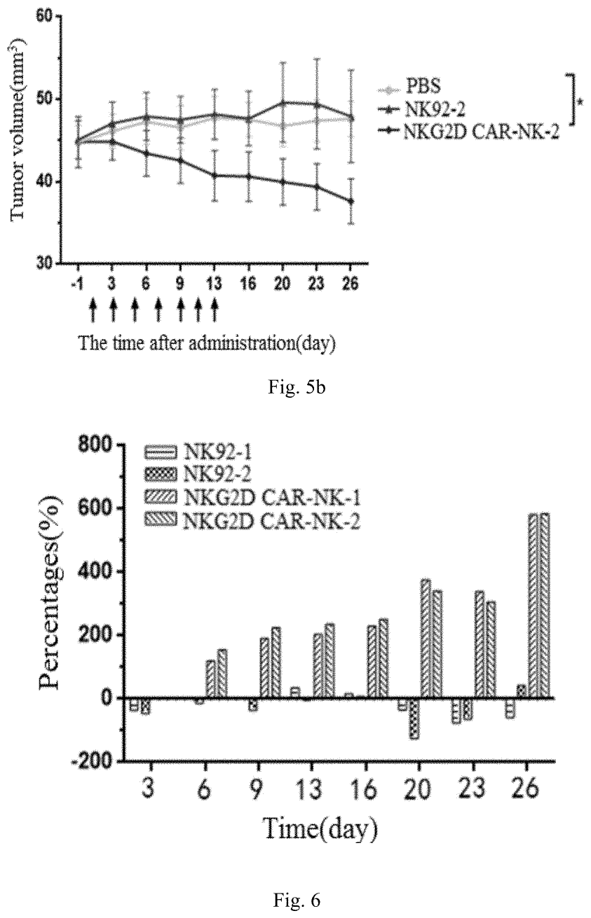

in the above-mentioned FIG. 5a to FIG. 8, PBS is the PBS administration group, NK92-1 is the administration group with a dosage of 5×10⁶ cells/mouse, NKG2D CAR-NK-1 is the administration group with a dosage of 2.5×10⁶ cells/mouse, NKG2D CAR-NK-2 is the administration group with a dosage of 5×10⁶ cells/mouse,

* Indicates P (significant difference)<0.05.

### DETAILED DESCRIPTION OF THE EMBODIMENTS

Unless otherwise defined, all technical and scientific terms used in the present invention have the same meaning as those generally understood by a person of ordinary skilled in the art.

Specifically, the nucleotide sequence for encoding fusion protein NKG2D-CD8αhinge-CD8™-4-1BB-CD3ζ in the present invention is any DNA sequence that can encode the fusion protein; preferably, the sequence is SEQ ID NO: 2 or its complementary sequence. On the other hand, the nucleotide sequence for encoding fusion protein NKG2D-CD8αhinge-CD8™-4-1BB-CD3ζ in the present invention can be the polynucleotide or its complementary sequence which can hybridize with the nucleotide sequence of SEQ ID NO:2 under stringent condition, and can encode the fusion protein.

The term of “stringent condition” described in this paper can be selected from low stringent condition, moderate stringent condition and high stringent condition. Illustratively, “low stringent condition” can be the conditions of 30° C., 5×SSC, 5× Denhardt solution, 0.5% SDS and 52% formamide; “moderate stringent condition” can be the conditions of 40° C., 5×SSC, 5× Denhardt solution, 0.5% SDS and 52% formamide; “high stringent condition” can be the condition of 50° C., 5×SSC, 5× Denhardt solution, 0.5% SDS and 52% formamide. The skilled in the art should understand that the higher the temperature is, the more highly homologous polynucleotides can be obtained. Besides, the skilled in the art can choose the factors which can affect the stringency of hybridization, such as temperature, probe concentration, probe length, ionic strength, time and salt concentration, to achieve the corresponding stringency.

Besides, the hybridizable polynucleotides can also be the polynucleotides which has the identity of about 60% or more, 70% or more, 71% or more, 72% or more, 73% or more, 74% or more, 75% or more, 76% or more, 77% or above, 78% or above, 79% or above, 80% or above, 81% or above, 82% or above, 83% or above, 84% or above, 85% or above, 86% or above, 87% or above, 88% or above, 89% or above, 90% or more, 91% or more, 92% or above, 93% or more, 94% or more, 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, 99.9% or more to polynucleotide encoding sequence number 6 when calculated using default parameters set by the homologous search softwares such as FASTA or BLAST or the like.

The identity of nucleotide sequences can be determined by using algorithm rule BLAST of Karlin and Altschul (Proc. Natl. Acad. Sci. USA 87: 2264-2268,1990; Proc. Natl. Acad. Sci. USA 90:5873,1993). The softwares of BLASTN and BLASTX based on the algorithm rule BLAST have been developed (Altschul SF, et al: J Mol Biol 215: 403, 1990). When analyzing the base sequence using BLASTX, the parameters can be exemplarily set as score=100 and word length=12; besides, when analyzing the amino acid sequence using BLASTX, the parameters can be exemplarily set as score=50 and word length=3; when using softwares of BLAST and Gapped BLAST, parameters can be set as default.

Unless otherwise specified, “encoding nucleotide” includes all nucleotide sequences that are degenerate versions of each other and can encode the same amino acid sequence. The nucleotide sequence for encoding protein can contain introns.

The term “lentivirus” refers to the genus of retroviruses, it can effectively infect non-cyclical and mitotic cells. They can transmit significant amounts of genetic information into DNA of host cells, so that they are one of the most effective methods of gene delivery vectors.

The term “promoter” is defined as a DNA sequence needed to start the specific transcription of polynucleotide sequences, which is identified by organelle or is to guide organelle.

The term “specific binding” means recognizing specific antigens but basically not recognizing or binding other molecules in the sample.

The term “carrier” or “vector” is a physical composition, it includes isolated nucleic acids, and can be used to transfer isolated nucleic acids into a cell. Many vectors which have been known in this field, including, but not limited to, linear polynucleotides, and the polynucleotides, plasmids and viruses related to ions or amphiphilic compound. Accordingly, the term “carrier” or “vector” includes autonomously replicating plasmids or viruses. The term should also be interpreted as including non-plasmid or non-viral compounds that facilitate transferring nucleic acids into cells, such as polylysine compounds and liposomes and the like. Examples of viral vectors include, but not limited to, adenovirus vectors, adeno-associated virus vectors, retroviral vectors, and so on.

The term “cancer” is defined as a disease characterized by rapid and uncontrolled growth of aberrant cells. Cancer cells can spread locally or spread to other parts of the body through blood flow and lymphatic system. Examples of various cancers include, but not limited to, breast cancer, colorectal cancer, liver cancer, lung cancer and so on.

As used in this text, “include”, “contain”, “comprise” or “characterized in” are synonymous, and are inclusive or open, and do not exclude other elements or methods or steps that are not stated. Any expression of the term “include” in this paper, especially when describing the methods, uses or products of the present invention, should be understood as including those products, methods and uses which are basically or totally composed of the components or elements or steps. The illustrative description in the present invention can be practiced properly without any one or more components or one or more restrictions which are not specifically described in this paper.

The terms and expressions used in this paper are used as descriptive rather than restrictive terms, and it is not expected to exclude any equivalents of the illustrated or described characteristics or their parts in the use of such terms and expressions, but it should be recognized that various modifications are acceptable within the scope of the present invention. Therefore, it should be understood that although the present invention has been specifically disclosed through preferred embodiments and optional features, the skilled in the art can adopt the modifications and variations of the concepts disclosed in this paper, and such modifications and variations are regarded as within the scope defined by the additional claims of the present invention.

The English names in this paper are not case sensitive, the terms of NKG2D CAR-NK, NKG2D-CAR NK both share the same meaning; the terms of NKG2D CAR-NK and NKG2D-CAR NK share the same meaning as CAR-NK cell of the ligands of NKG2D molecule; the terms of NK-92 and NK92 share the same meaning as NK92 cells, CD8™represents the transmembrane domain.

The term of “NK” described in the present invention are human normal NK cells or NKT cells, or NK cell lines that include NK-92 cells, YT cells, NKL cells, HANK-1 cells, NK-YS cells, KHYG-1 cells, SNK-6 cells and IMC-1 cells, and so on. In the specific embodiment of the invention, NK-92 cell is taken as an example.

For a clearer explanation of the invention, it is explained in detail in combination with the following embodiments. But these embodiments are merely exemplary descriptions of the invention and not intended to limit the present invention.

Source materials in the following examples: NK-92 cells (CC® CRL-2407), lung cancer cells A549, breast cancer MDMB-231 cells, colorectal cancer cells SW480, breast cancer MCF-7 cells, HCC1187 cells, and lung cancer cells H1299 were all obtained from Cell Resource Center, Shanghai Institute for Biological Sciences, Chinese Academy of Sciences.

NPG mice were obtained from Beijing Weitong Lihua Laboratory Animal Technology Co., Ltd., SPF grade, female, 5-6 weeks, body weight: 18-20 g, ±20%.

### Example 1 Preparation of Lentiviral Vector

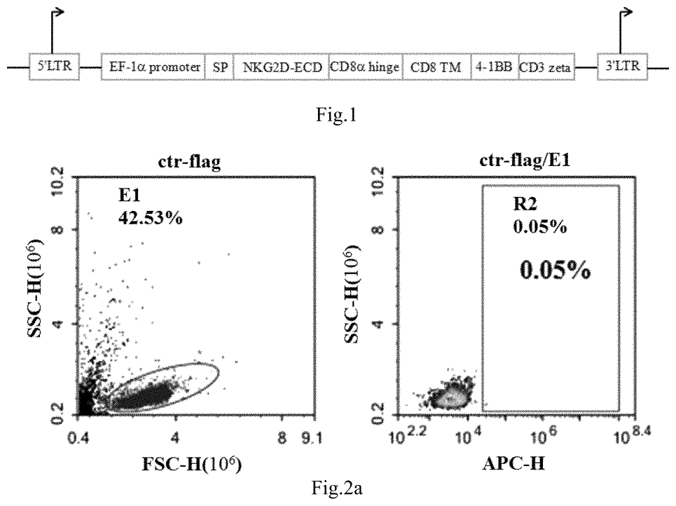

The sequence of NKG2D-CD8ahinge-CD8™-4-1BB-CD3ζ fusion gene (its amino acid sequence is shown in SEQ ID NO:1 and its gene sequence is shown in SEQ ID NO:2) is synthesized, and the sequence of NKG2D-CD8ahinge-CD8™-4-1BB-CD3ζ fusion gene was transformed and ligated into PRRSLIN vector by enzyme digestion, wherein the upstream of the gene was EF-1α promoter. The vector was transformed into Stb13 Escherichia coli strain, which was screened by ampicillin to obtain positive clones. Then plasmid was extracted and the clone was identified by enzyme digestion, so that PRRLSIN-NKG2D lentiviral vector was obtained (as shown in FIG. 1).

### Example 2 Preparation of Lentivirus

(1) 24 hours before transfection, 293T cells were inoculated into 15 cm petri dishes at about 8×10⁶ per dish. Ensure that the convergence degree of 293T cells was about 80% and the 293T cells were evenly distributed in petri dishes during the transfection.

(2) Preparation of solution A and solution B

Solution A: 6.25 mL of 2× HEPES buffered solution (the effect is better when the quantity is a package of 5 dishes together)

Solution B was a mixture by adding the following plasmids respectively: 112.5 μg PRRLSIN-NKG2D (target plasmid); 39.5 μg pMD2.G(VSV-G envelop); 73 μg pCMVR8.74 (gag, pol, tat, rev); 625 μl 2M calcium ion solution. The total volume of solution B was 6.25 mL.

Solution B was mixed completely, and dropwise added into solution A while gently swirling solution A, so that a mixture of solution A and B was obtained, which was then rested to stand for 5 to 15 minutes. The mixture of solution A and B was gently swirled and dropwise added into a petri dish containing 293T cells. The obtained petri dish was gently shaken back and forth to evenly distribute the mixture of DNA and calcium ions. Then the petri dish was incubated for 16-18 hours in incubator (without being rotated). The medium was replaced by fresh medium and continued the cultivated. The supernatants containing virus was collected after 48 hours and 72 hours separately, and the supernatants was centrifuged at 500 g for 10 min at 25° C. and then filtered by PES membrane (0.45 μm), and the filtered supernatants of lentivirus was obtained. Ultra-clear SW28 centrifuge tubes (by the manufacture of BECKMAN COULTER) were disinfected with 70% ethanol and then sterilized under ultraviolet light for 30 min. The filtered supernatants of lentivirus were transferred into the prepared centrifuge tube which had been carefully laid a layer of 20% sucrose (1 mL sucrose per 8 mL supernatant) at the bottom. The liquid was balanced with PBS buffer and then centrifuged at 25000 rpm (82,700 g) at 4° C. for 2 hours. The centrifuge tubes were taken out carefully, and the supernatants were poured out, and then centrifuge tubes were inverted to remove the residual liquid. Then the centrifuge tubes were added by 100 μl PBS, sealed, stood at 4° C. for 2 hours, swirled once every 20 minutes, and finally centrifuged at 500 g for 1 min (25° C.) to collect the supernatant containing virus, and obtained lentivirus. The collected supernatant was cooled on ice and then stored at −80° C.

### Example 3 Preparation of NKG2D CAR-NK Cells

The density of NK-92 cells was adjusted to 2×10⁵ /mL to 3×10⁵/mL. The virus obtained from example 2 was added to NK-92 cells according to the volume ratio (V/V) of virus: cell culture medium=1:5, while 8 μg/mL polyamine was added at the same time. 4 hours later, equivalent amount of fresh complete medium was added to adjust the cell density to 1×10⁵/mL for further cultivation. The next day, all cells were centrifuged and added by fresh medium for further cultivation. Fresh medium was added every 1 to 2 days to maintain cell density at 2×10⁵/mL to 3×10⁵/mL. CAR antibody staining was performed after 72 hours, and the positive cells of NKG2D CAR NK-92 were sorted by flow cytometry for culture expansion. The color change, cell density and cell morphology of the medium were observed daily and recorded accordingly.

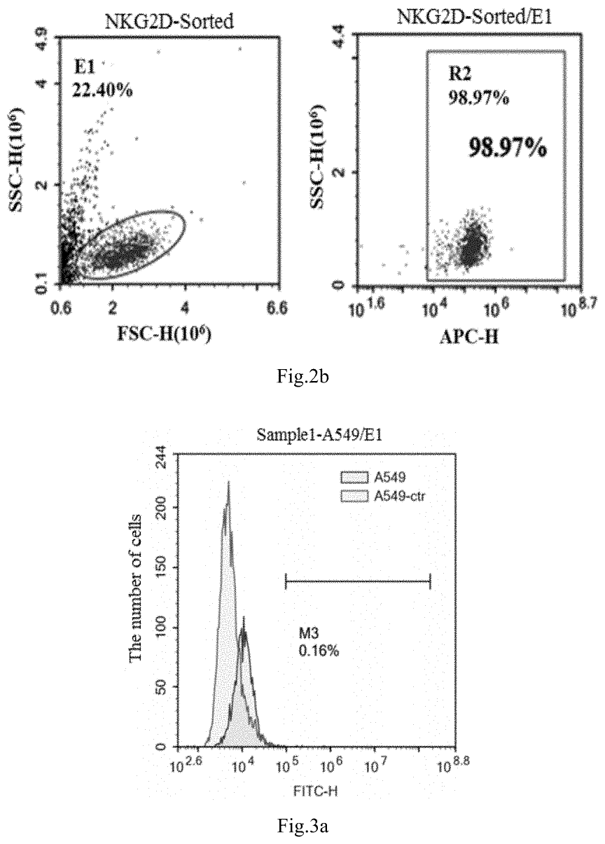

The positive rate of NKG2D CAR NK-92 cells were detected with flow cytometry, the detected results are shown in FIG. 2a and FIG. 2b. FIG. 2a is the control group, which are NK92 cells without CAR molecule; FIG. 2b is the experimental group, which are NK92 cells with CAR molecule. In FIG. 2a and FIG. 2b, the ordinate SSC-H represents the lateral scattering value of flow cytometry, the abscissa FSC-H represents the forward scattering value of flow cytometry, and the two parameters are mainly used to delineate two groups of live cells for analysis, which are shown as elliptic delineation in the figure; APC-H represents the fluorescence intensity stained with antibodies, The stronger the fluorescence intensity, which represents the greater the positive rate of NKG2D CAR NK-92 cells compared to the control group. It can be seen from FIG. 2a and FIG. 2b that the signal value of the fluorescence label increased significantly, indicating that CAR molecules were successfully expressed by NK92 cells. The positive rate of CAR-NK92 is 98.97%.

### Example 4 Evaluation of the Specific Lysis Activity of NKG2D CAR-NK Cells on Tumor in Vitro

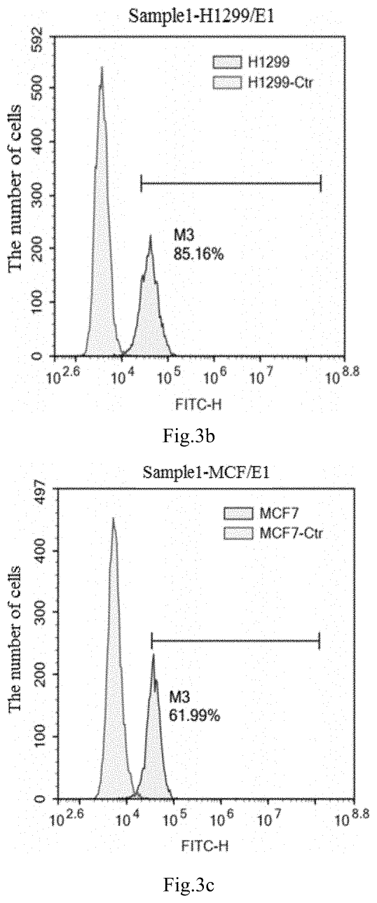

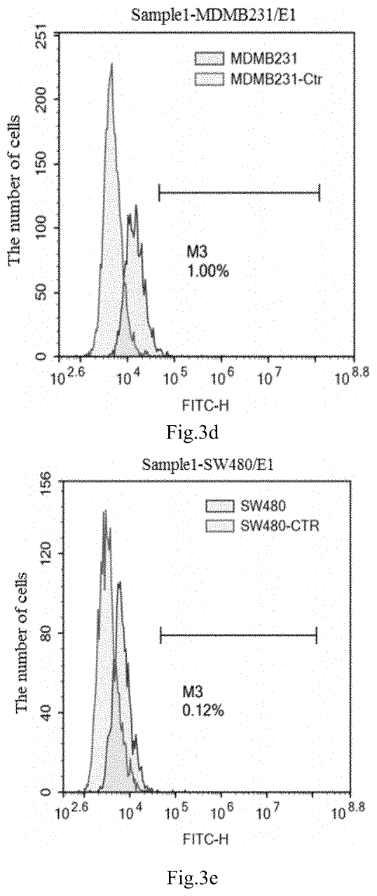

In order to determine the tumor cells used for the detection of NKG2D CAR NK-92, the expression of NKG2D ligand MIC-A and MIC-B molecules indifferent tumor cells were detected using conventional flow cytometry method. Wherein, the selected tumor cells were lung cancer cell A549, breast cancer cell MDMB-231, colorectal cancer cell SW480, breast cancer cell MCF-7, and lung cancer cell H1299, and their experimental results were shown in FIG. 3a to FIG. 3e.

FIG. 3a is an experimental result of lung cancer cell A549, FIG. 3b is an experimental result of lung cancer cell H1299, FIG. 3c is an experimental result of breast cancer cell MCF-7, FIG. 3d is an experimental result of breast cancer cell MDMB-231, FIG. 3e is an experimental result of colorectal cancer cell SW480.

It can be seen from FIG. 3a to FIG. 3e, the NKG2D ligand MIC-A or MIC-B molecules are low expression in lung cancer cell A549, breast cancer cell MDMB-231, and colorectal cancer cell SW480, the rate of their expression is 0.19%, 1%, and 0.12% separately; that are moderate expression in breast cancer cell MCF-7, and their positive rate is 61.99%; that are highly expression in lung cancer cell H1299, and their positive rate is 85.16%.

The specific lysis activity of NKG2D CAR-NK cells on various tumor cells was detected using CCK-8 method (reference: Human Leukocyte Antigen-G Inhibits the Anti-Tumor Effect of Natural Killer Cells via Immunoglobulin-Like Transcript 2 in Gastric Cancer, Rui Wan Zi-Wei Wang Hui Li, et al.). The operation method of the experiment is as follows:

1) The above-mentioned five tumor cells were evenly spread in 24 wells one day in advance, which was followed by adhesive.

2) On the next day, the NKG2D CAR-NK cells were spread in 24 cells(which is 20000 cells and 40000 cells respectively)with the ratio of effector cells to target cells=0.5:1, or 1:1, its total system is 1 mL, and were incubated for 4 hours.

(3)After 4 hours, the medium was discarded, and rinsed gently with PBS twice, and 200 ul of CCK8 reagent was added, and followed being incubated at 37° C. for Ho 4 hours.

(4) The specific lysis activity was measured at 450 nm.

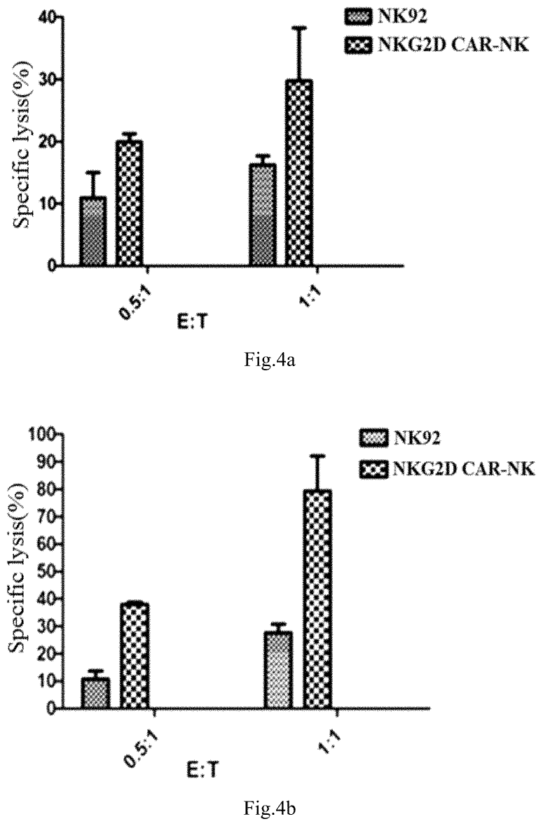

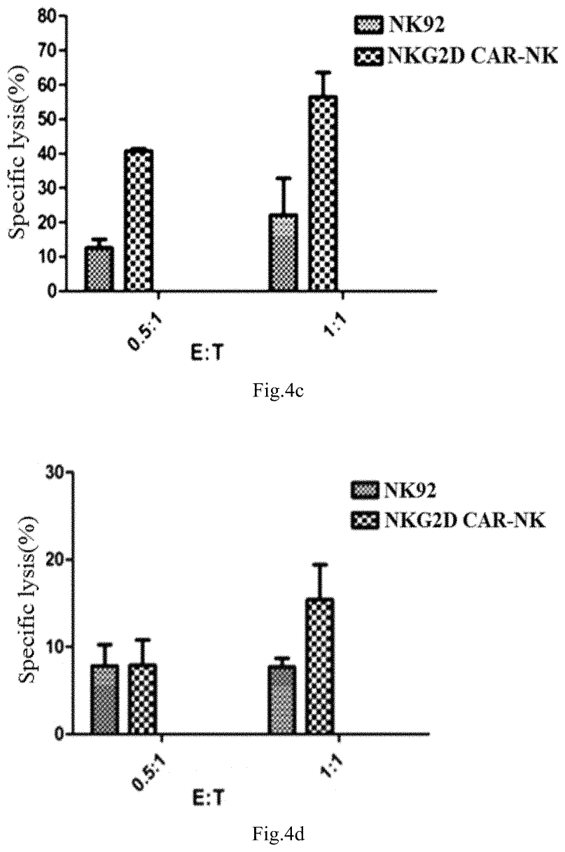

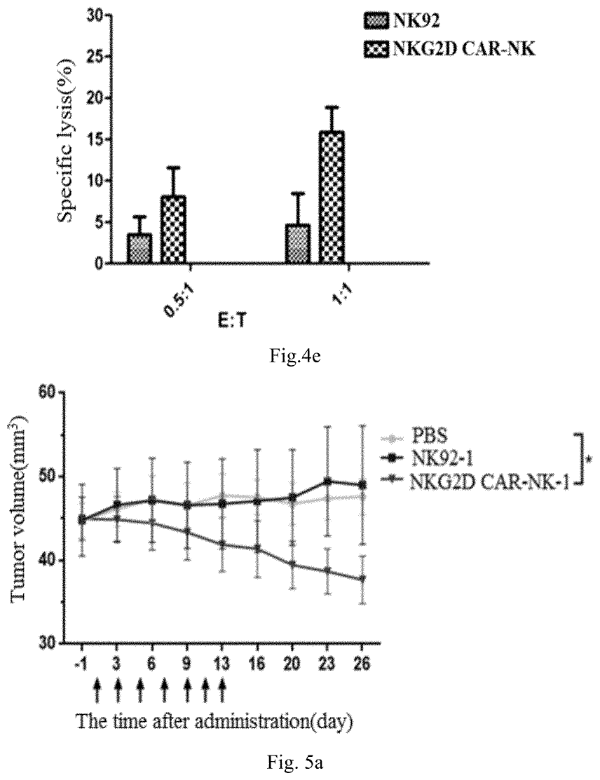

The evaluation results of the specific lysis activity of the NKG2D CAR-NK cells on tumor cells in vitro are shown in the following table 1 and FIGS. 4a to 4e.

TABLE 1
the specific lysis activity of the NKG2D
CAR-NK cells on tumor cells in vitro

| | |
|---|---|
| effective target | effective target |
| ratio is 0.5:1 | ratio is 1:1 |

| | | NKG2D | | NKG2D |
|---|---|---|---|---|
| | NK-92 | CAR-NK | NK-92 | CAR-NK |
| cells | group | group | group | group |
| A549 | 11% | 21% | 18% | 30% |
| H1299 | 12% | 39% | 28% | 80% |
| MCF-7 | 14% | 43% | 20% | 56% |
| MDMB-231 | 7% | 7% | 7% | 14% |
| SW-480 | 3% | 8% | 5% | 16% |

In FIGS. 4a to 4e, the ordinate represents specific lysis activity, the abscissa represents the ratio of effective target ratio. Wherein FIG. 4a is an experimental result of lung cancer cell A549, FIG. 4b is an experimental result of lung cancer cell H1299, FIG. 4c is an experimental result of breast cancer cell MCF-7,FIG. 4d is an experimental result of breast cancer cell MDMB-231, FIG. 4e is an experimental result of colorectal cancer cell SW480.

As shown in table 1 and FIGS. 4a to 4e, on the H1299 cell with highly expressing of NKG2D ligands, the specific lysis activity of the NKG2D CAR-NK is superior to the common NK-92 cells under the condition of effective target ratio of 0.5:1 and 1:1, wherein the specific lysis activity can reach 80% under the condition of effective target ratio 1:1. On the MCF-7 with moderately expressing of NKG2D ligands, the specific lysis activity of the NKG2D CAR-NK is superior to the common NK-92 cells, and the specific lysis activity can reach 56% under the condition of effective target ratio 1:1. On the other tumor cells with lowly expressing of NKG2D ligands, the specific lysis activity of the NKG2D CAR-NK is also superior to the common NK-92 cells. That is, the specific lysis activity of NKG2D CAR-NK is better than that of common NK-92 cells, but the specific lysis activity on cells with lowly expressing of NKG2D ligands is not as obvious as that of tumor cells with highly expressing of this ligands.

### Example 5 Evaluation of Specific Lysis Activity of NKG2D CAR-NK Cells on Tumor in Vivo

HCC1187 cells were continuously incubated, and before 10 generations, the cells in the logarithmic growth phase were collected and inoculated in the back of NPG mice by subcutaneous injection. Each point is inoculated with about 2×10⁶ cells, and the inoculation volume is about 100 μL.

1. Specific administration design: when the tumor volume reaches 40 to 50 mm³, the mice were divide into groups, the drug was resuspended in PBS to the concentration for designing dosing administration, and administrated according to the set group in the corresponding dosing mode. The dosing group includes NKG2D CAR-NK cell group, PBS control Group and NK-92 group, which were administrated at 2.5×10⁶/time and 5×10⁶/time, respectively, and in which two parallel trials were designed.

The specific administration groups are shown in the following table2:

TABLE 2

| | | Number | | | | | |
|---|---|---|---|---|---|---|---|
| | | of | Dosage | Administration | Drug | Administration | Administration |
| Drug/group | Tumor | animals | (cell/mouse) | volume | concentration | route | mode |
| PBS | HCC1187 | 6 | — | 250 μl | — | i.v. | Once every |
| NK92(NK92-1) | | 6 | 2.5 × 10⁶ | | 1 × 10⁷/ml | | other day |
| NK92(NK92-2) | | 6 | 5 × 10⁶ | | 2 × 10⁷/ml | | for a total of |
| NKG2D CAR- | | 6 | 2.5 × 10⁶ | | 1 × 10⁷/ml | | 7 times |
| NK(NKG2D | | | | | | | |
| CAR-NK-1) | | | | | | | |
| NKG2D CAR- | | 6 | 5 × 10⁶ | | 2 × 10⁷/ml | | |
| NK(NKG2D | | | | | | | |
| CAR-NK-2) | | | | | | | |

2. The calculation of growth curve and inhibition rate of tumor in mice

After administration, observed the appearance and behavior of animal NPG mice, measured their weight, and calculated their tumor growth inhibition rate.

tumor volume(V, mm³), its calculation formula: V=(length×width²)/2.

Tumor volume ratio of treatment group/control group(T/C, %)=(Td−T0)/(Cd−C0)×100%, Td and Cd respectively represent as the tumor volume of the administration group and control group at the last measurement, T0 and C0 respectively represent as the tumor volume of the administration group and the control group being divided.

Tumor growth inhibition rate(TGI, %), its calculation formula: TGI=(1−T/C)×100%, the specific inhibitory effects are shown in the follow Table 3, and FIGS. 5a to b and FIG. 6.

TABLE 3

| | The | | | | | |
|---|---|---|---|---|---|---|
| | number of | Tumor volume | | Tumor growth inhibition rate(TGI) | | |
| Group | animals | V₀(mm³)^{a} | Vₜ(mm³)^{a} | TIC(%) | TGI(%) | P^{b} |
| PBS | 6 | 44.80 × 5.05 | 47.63 × 5.20 | — | — | — |
| NK92-1 | 6 | 44.82 × 10.50 | 49.00 × 17.33 | 158.61% | −58.61% | 0.856 |
| NK92-2 | 6 | 45.12 × 5.64 | 47.93 × 13.71 | 56.93% | 43.07% | 0.962 |
| NKG2D CAR- | 6 | 44.98 × 6.30 | 37.70 × 6.92 | −483.00% | 583.00% | 0.018 |
| NK-1 | | | | | | |
| NKG2D CAR- | 6 | 44.83 × 7.55 | 37.65 × 6.66 | −485.77% | 585.77% | 0.016 |
| NK-2 | | | | | | |

Note: a represents Mean±SD; b represents P value compared with Solution; V0 represents tumor volume before administration and Vt means tumor volume after administration.

The results of the tumor growth curve are shown in FIG. 5a to b, It can be seen from the figures that the tumor volume is significantly smaller than that of the NK92 group and PBS group after NKG2D CAR-NK is used to kill HCC1187 tumor-forming mice. The results of the tumor growth inhibition rate are shown in FIG. 6, there is obviously tumor inhibition effect in the NKG2D CAR-NK-1group and NKG2D CAR-NK-2 group, which is gradually increased from the 6th day after administration until the end of the experiment. However, there is always not obvious tumor inhibition effect in the NK92-1 group and NK92-2 group.

3. Changes in tumor weight

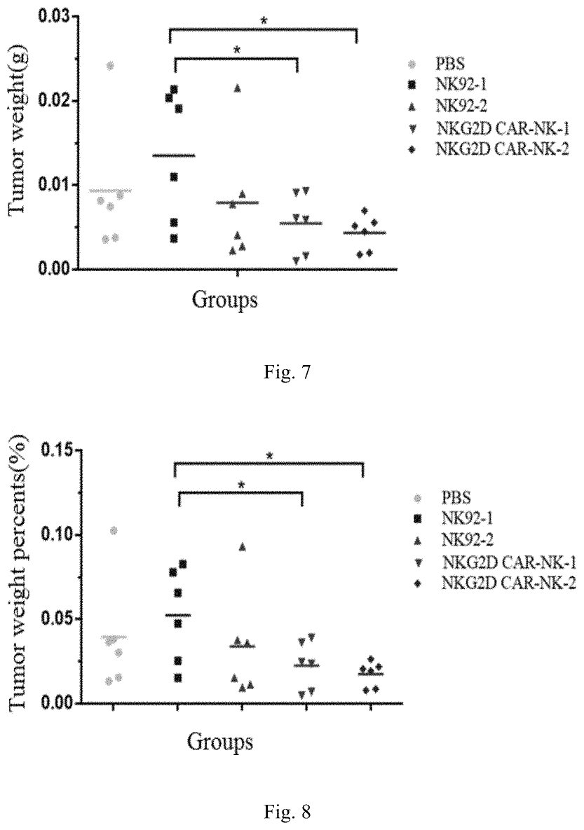

After the test, euthanized the NPG mice, and removed the tumor tissue of each group of mice, photographed and weighed. The tumor weight and the percentage of the tumor weight in the body weight (% of tumor weight) are shown in FIGS. 7 to 8. From the tumor weight and the percentage of tumor weight in the body weight in each group, the tumor weight and tumor weight percentage of NK92-1 group were the largest. The tumor weight and the percentage of tumor weight decreased gradually in the order of PBS group, NK92-1, NKG2D CAR-NK-1 group and NKG2D CAR-NK-2, and the consistency was good compared with the results of the tumor volume measured 26 days after the administration.

The above experimental results show that the specific lysis and tumor inhibition effect of the NKG2D CAR-NK cells of the present invention are significantly better than that of the NK92 group, and the NKG2D CAR-NK cell constructed by the present invention significantly improves its targeting by increasing the cooperation of CAR and NKG2D receptors, which greatly improves its specific lysis and tumor inhibition effect, and can better resist tumors.

The above mentioned is only preferable embodiments of the present invention, and cannot be used to restrict the invention. Any modification, equivalent replacement and the like within the spirit and principles of the present invention should be included in the scope of protection of the present invention.

What is claimed is:

1. A chimeric antigen receptor, which comprises an antigen binding domain, a transmembrane domain and a costimulatory signal transduction region, and the antigen binding domain can specifically bind to tumor specific antigen NKG2D ligands and can activate NK cells through the transmembrane domain and the costimulatory signal transduction region,
the antigen comprises NKG2D, and the NKG2D can specifically bind to NKG2D ligands.

2. The chimeric antigen receptor of claim 1, wherein the NKG2D ligands are selected from one or more of major histocompatibility complex class I (MHC-I), MIC-A and MIC-B, and/or, the transmembrane domain is selected from one or more of CD28, CD3ε, CD45, CD4, CD5, CD8, CD9, CD16, CD22, CD33, CD37, CD134, CD137, ICOS and CD154, preferably, the transmembrane domain is CD8 transmembrane domain, and/or,
the costimulatory signal transduction region contains the intracellular domain of costimulatory molecule, which is selected from one or more of CD3ζ, CD3γ, CD3δ, CD3ε, CD5, CD22, CD79a, CD79b, CD66d, CD2, CD4, CD5, CD28, CD134, CD137, ICOS, CD154, 4-1BB and OX40.

3. The chimeric antigen receptor of claim 2, wherein the costimulatory signal transduction region contains the intracellular domains of 4-1BB and CD3ζ.

4. The chimeric antigen receptor of claim 3, wherein the chimeric antigen receptor is a fusion protein with a structure of NKG2D-CD8αhinge-CD8™-4-1BB-CD3ζ, the amino acids sequence of the fusion protein NKG2D-CD8αhinge-CD8™-4-1BB-CD3ζ is shown in SEQ ID NO: 1 or homologous sequence thereof and so on,
the homologous sequence has the identity of about 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, 99.9% or more to the original sequence.

5. A NKG2D CAR-NK cell expressing a chimeric antigen receptor, wherein the chimeric antigen receptor comprises an antigen binding domain, a transmembrane domain and a costimulatory signal transduction region, and the antigen binding domain can specifically bind to tumor specific antigen NKG2D ligands and can activate NK cells through the transmembrane domain and the costimulatory signal transduction region, the antigen comprises NKG2D, and the NKG2D can specifically bind to NKG2D ligands.

6. The NKG2D CAR-NK cell of claim 5, wherein the NKG2D ligands are selected from one or more of major histocompatibility complex class I (MHC-I), MIC-A and MIC-B, and/or, the transmembrane domain is selected from one or more of CD28, CD3ε, CD45, CD4, CDS, CD8, CD9, CD16, CD22, CD33, CD37, CD134, CD137, ICOS and CD154, preferably, the transmembrane domain is CD8 transmembrane domain, and/or,
the costimulatory signal transduction region contains the intracellular domain of costimulatory molecule, which is selected from one or more of CD3ζ, CD3γ, CD3δ, CD3ε, CD5, CD22, CD79a, CD79b, CD66d, CD2, CD4, CDS, CD28, CD134, CD137, ICOS, CD154, 4-1BB and OX40.

7. The NKG2D CAR-NK cell of claim 6, wherein the costimulatory signal transduction region contains the intracellular domains of 4-1BB and CD3ζ.

8. The NKG2D CAR-NK cell of claim 7, wherein the chimeric antigen receptor is a fusion protein with a structure of NKG2D-CD8αhinge-CD8™-4-1BB-CD3ζ, the amino acids sequence of the fusion protein NKG2D-CD8αhinge-CD8™-4-1BB-CD3ζ is shown in SEQ ID NO: 1 or homologous sequence thereof and so on,
the homologous sequence has the identity of about 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, 99.9% or more to the original sequence.

9. The NKG2D CAR-NK cell of claim 8, wherein the encoding nucleotide sequence of the fusion protein NKG2D-CD8™αhinge-CD8-4-1BB-CD3ζ is shown in SEQ ID NO: 2 or homologous sequence thereof and so on,
and the homologous sequence has the identity of about 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, 99.9% or more to the original sequence.

10. The NKG2D CAR-NK cell of claim 5, wherein the can effectively destroy or kill lung cancer cell line, colorectal cancer cell line, or breast cancer cell line.

11. A pharmaceutical composition comprising the NKG2D CAR-NK cell of claim 5.

12. The pharmaceutical composition of claim 11, wherein the effector-to-target ratio of NKG2D CAR-NK cells to tumor cells in the pharmaceutical composition is 0.5:1 to 1:1.

13. The pharmaceutical composition of claim 11, wherein also comprises optional pharmaceutically acceptable adjuvants.

14. The pharmaceutical composition of claim 11, wherein the dosage form of the pharmaceutical composition is aqua.

15. A method for treating and/or preventing cancer using the NKG2D CAR-NK cell of claim 5 wherein the method includes administering an effective amount of drug containing NKG2D CAR-NK cells to patients needing treatment.

16. The method of claim 15, wherein the cancer is a tumor with highly expressing of NKG2D ligands and related diseases.

17. The method of claim 15, wherein the cancer is lung cancer, breast cancer, or colorectal cancer.

18. The method of claim 15, wherein the dosage of NKG2D CAR-NK cells is 1×10⁶ cells/time to 10×10⁶ cells/time.

19. The method of claim 15, wherein the administration mode of the drug containing NKG2D CAR-NK cells is intratumoral injection, intravenous injection, intrathoracic injection or local intervention.

20. The method of claim 19, wherein the administration mode of the drug containing NKG2D CAR-NK cells is intravenous injection.
